(19) United States
(12) Patent Application Publication
(10) Pub. No.: US 2012/0245042 A1
(43) Pub. Date: Sep. 27, 2012

(54) DEBUBBLER FOR MICROFLUIDIC SYSTEMS

(75) Inventors: Changchun Liu, Philadelphia, PA (US); Haim H. Bau, Swarthmore, PA (US); Jason Alan Thompson, Quaker Street Village, NY (US)

(73) Assignee: THE TRUSTEES OF THE UNIVERSITY OF PENNSYLVANIA, Philadelphia, PA (US)

(21) Appl. No.: 13/398,192

(22) Filed: Feb. 16, 2012

Related U.S. Application Data
(60) Provisional application No. 61/452,416, filed on Mar. 14, 2011.

(51) Int. Cl. B01D 19/00; G01N 30/96; C12M 1/34; B81B 3/00; C40B 30/00

(52) U.S. Cl. 506/7; 96/6; 95/46; 435/287.2; 422/69

(57) ABSTRACT

Provided are robust, passive, membrane-based debubblers that are readily incorporated into microfluidic devices for rapid degassing. Also provided are methods of degassing fluid disposed within fluidic systems.

### CROSS REFERENCE TO RELATED PATENT APPLICATION

The present application claims priority to U.S. Application No. 61/452,416, “Debubbler For Microfluidic Systems,” filed on Mar. 14, 2011, and incorporated herein by reference in its entirety for any and all purposes.

### STATEMENT OF GOVERNMENT RIGHTS

This invention was made with government support under grant U01DE017855 awarded by the National Institutes of Health. The government has certain rights in this invention.

### TECHNICAL FIELD

The present invention relates to the field of fluid mechanics and to the field of microfluidic devices.

### BACKGROUND

In some microfluidic applications, bubbles present in the system can negatively affect device operation and experimental outcomes. Bubbles may be formed at interconnects, and can be introduced when switching flow streams during sequential flow. Bubbles may also be produced during heating and during certain reactions.

Air bubbles in microfluidic devices can obstruct fluidic paths and distort flows, damage cells at a liquid-gas interface, reduce PCR amplification efficiency, and interfere with bead array-based assays. A great deal of time and skill is required to operate and fill these devices without bubbles, and this time commitment can decreases the overall efficiency of the systems. Accordingly, there is a need in the art for debubbler units suitable for integration into microfluidic systems.

### SUMMARY

To address these challenges, provided are robust, systems for rapid and efficient removal of air bubbles from liquid solutions even when the liquids contain various surfactants. In some embodiments, the devices resemble a normally closed valve that opens when subjected to pressure from a working liquid.

First provided are debubblers. The debubblers include a substrate having at least one of a first fluid channel and a second fluid channel formed therein; and a flexible gas-permeable hydrophobic membrane sealing an outlet of the first fluid channel, the first and second fluid channels being in fluid communication with one another when the flexible gas-permeable hydrophobic membrane is in a deflected state.

Also provided are methods of degassing a fluid. These methods include exerting a fluid contained within a first fluid channel against a gas-permeable membrane sealing the first fluid channel so as to discharge a gas disposed in the first fluid channel through the membrane while the membrane remains essentially stationary; exerting the fluid against the membrane so as to deflect the membrane, the deflection of the membrane placing the first fluid channel into fluid communication with a second fluid channel such that the fluid flows into the second fluid channel.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1: A schematic depiction of an exemplary debubbler and its degassing principle: (a) Initial, closed state before liquid enters the debubbler. (b) Open state with liquid flowing thorugh the debubbler. The inset shows an air—liquid meniscus pinned at the pore's entrance; surface tension maintains the pressure difference across the meniscus and prevents liquid from leaking through the pore. (c) Closed state with air bubble in the debubbler. The bubble was forced to discharge through the hydrophobic pore of the PTFE membrane.

FIG. 2: A bead array-based microfluidic cassette with integrated debubbler. (a) Exploded view of integrated cassette. The cassette consists of a top PMMA film, porous membranes within double-sided tape, a PMMA cassette body, agarose beads, black tape, and a bottom PMMA film. All microstructure features including nozzles, microchannels, and the 5×3 well array are milled in the PMMA cassette's body. (b) A photograph of the assembled cassette.

FIG. 3: (a) A sequence of images illustrating the bubble removal process from DI water in the membrane-based debubbler. Bubbles traveling from left to right at a flow rate of 200 μl/min are completely removed from the liquid stream through the porous membrane. (i) 0 s. A bubble enters the debubbler. (ii) 0.1 s. The bubble enters the membrane region. (iii) 0.2 s. The bubble is vented. (iv) 0.3 s. The liquid downstream of the membrane is completely free of bubbles. (b) The flow rates of DI water and PBS blocking buffer through the debubbler as functions of liquid pressure (p₁−p₀)) at the debubbler's inlet. The error bars correspond to the scatter of the data obtained in three experiments.

FIG. 4: Detection of PCR amplicons of B. Cereus genomic DNA: (a) A fluorescent image of three streptavidin-coated beads at different DNA concentrations in the integrated microfluidic debubbler cassette. Groups 1, 2, 3, 4 and 5 correspond, respectively, to template masses of 10, 1, 0.1, 0.01 and 0 ng (negative control) of DNA. (b) Measured intensity of agarose beads at different PCR amplicon concentrations obtained from 0 to 10 ng template. The various samples are cross-referenced with (a). The error bars correspond to the scatter of the data obtained in six agarose beads. (c) Agarose gel (2.0%) electrophoresis images of PCR products amplified from B. Cereus genomic DNA. The various lanes are cross-referenced with (a). Lane M is the DNA Marker VIII ladder.

FIG. 5: Cross-sectional view of the assembled microfluidic cassette.

FIG. 6: (a) Fluorescent image of an agarose bead in a microfluidic cassette integrated with a debubbler. No observable air bubble was trapped in the bead well. (b) Fluorescent image of an agarose bead in a microfluidic cassette without a debubbler. Air bubbles trapped in the bead well disturb DNA detection and subsequent fluorescent imaging. In contrast to the degassed case (a), the bead (outlined with a dashed line) is barely visible.

FIG. 7: Background fluorescent intensity as a function of the substrate material. (1) 0.8 mm-thick PMMA substrate with black tape; (2) 0.8 mm-thick PMMA substrate without tape; (3) 3 mm-thick PMMA substrate with black tape; and (4) 3 mm-thick PMMA substrate without tape. The photograph above the top of each column is a fluorescent image of the corresponding substrate material.

FIG. 8 A schematic depiction of the streptavidin-coated agarose bead-based assay. Biotin and dig labeled DNA amplicon first bind to the streptavidin-coated agarose bead through their biotin functionalization. Then, the anti-digoxigenin-fluorescein complex binds to the dig end of the DNA amplicon.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

First provided are debubblers. The debubblers may be present as modules that are inserted into or even machined within a fluidic system, or may be stand-alone devices. In some embodiments, debubblers according to the present disclosure are integrated into a fluidic system. The debubblers may be present at the inlet to the system, at the outlet of the system, or present at various locations within the system. The debubbler may be valved such that fluid being processed within a system is routed through the debubblers. Alternatively, the valve may be configured to have fluid bypass the debubblers. Debubblers may be disposed in series with one another or even in parallel with one another.

The debubbler suitably includes a substrate having at least one of a entry fluid channel and a exit fluid channel formed therein. A flexible gas-permeable hydrophobic membrane may be sealed to the substrate, and suitably seals an outlet of the entry (or first) fluid channel. The membrane is configured such that the first and second fluid channels are in fluid communication with one another when the membrane is in a deflected state.

The flexible, gas-permeable hydrophobic membrane is suitably porous. Polytetafluoroethylene. (PTFE), Polyvinylidene fluoride (PVDF), Polypropylene (PP), Polyethylene (PE), acrylic polymers, and the like. Other flexible materials coated with a hydrophobic coating are all suitable membrane materials. PTFE membranes—such as those available from the Sterlitech Corporation—are considered particularly suitable. The membrane is suitably constructed (or chosen) such that the membrane is essentially impermeable to liquids when the liquid pressure is below a certain threshold, as explained below in additional detail.

The entry and exit fluid channels may be formed in a single substrate. In alternative embodiments, the fluid channels are formed in different substrates. In such embodiments, the different substrates may be bonded to one another or placed adjacent to one another such that the membrane seals the fluid channels. Devices where the channels are formed in a single

The membrane is suitably attached to the substrate. This attachment may be by way of a glue, by tape, by ultrasonic or infrared welding, by thermal bonding, and the like. In some embodiments—described herein in further detail—the membrane is disposed within a layer of adhesive tape.

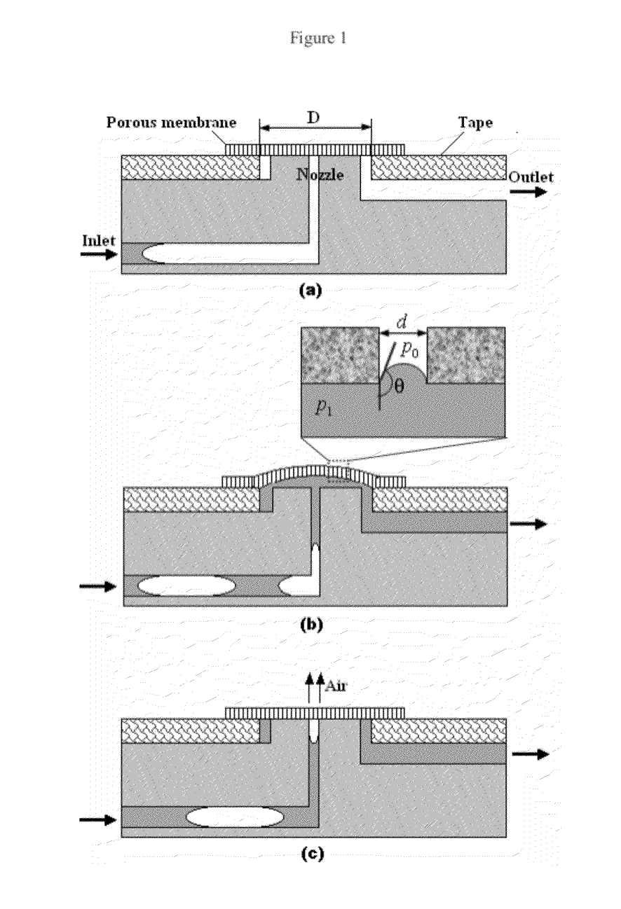

In some embodiments, the substrate includes a raised portion, a rim-like portion, or an island-like portion contacted by the flexible gas-permeable hydrophobic membrane. This is shown by FIG. 1, which illustrates the membrane contacting a raised or island-like portion of the substrate. At least one of the first fluid channel or the second fluid channel may be formed in the raised or island-like portion of the substrate, as shown in FIG. 1.

The membrane and substrate may be configured to as to define a gutter that is capable of fluid communication with the second fluid channel. The gutter may, as shown, encircle the raised rim portion of the substrate and act to “catch” fluid that exits the first fluid channel and then direct that fluid into the second channel. The substrate may also be essentially flat but also include a gutter that is formed (e.g., via etching, machining) in the substrate around the exit of the first fluid channel, which gutter is sealed by the membrane.

Such gutters are optional. In some embodiments, the membrane seals the first and second fluid channels; when the membrane is deflected by the pressure of the fluid in the first channel, the fluid that then exits the first channel fills the space beneath the now-deflected membrane and then flows into the second fluid channel.

A debubbler (membrane) may be in fluid communication with one, two, or more inlets. While FIG. 1 illustrates a single conduit (first conduit) that acts as an inlet to the membrane, the debubbler may have multiple inlets. Similarly, the debubbler may have multiple outlets; while FIG. 1 illustrates the membrane sealing one outlet, the membrane may in fact be in fluid communication with two or more outlets.

The substrate may be a polymer, a metal, a glass, a ceramic, silicon, and the like. PMMA polymer is considered suitable, as the material is well-characterized and is easily machined to feature channels, recesses, and the like. The substrate may be a multilayered structure, where different layers are shaped or stenciled so as to form channels, recesses, and the like. The substrate may also be injection molded. Polycarbonate, polystyrene, polymethylmethacrylate, polyethylene, polypropylene and the like are all suitable substrate materials. In some embodiments, the membrane is a part of the microfluidic device (e.g., a monolithinc pact) and can be formed during the fabrication process of the device as when the device is injection-molded. In some embodiments, the membrane is molded into the device as the device is fabricated. As one such example, the membrane may be placed into a mold and the other of the device (e.g., fluid channels) are molded in and around the membrane. In other embodiments, the membrane is bonded to the device.

The debubblers may include a connection to a fluid source (e.g., a reservoir) in fluid communication with the first fluid channel. A debubbler in module form may include an inlet port or other connector (e.g., a valve, Luer-lock™, or other like device) that places the debubbler module in fluid communication with a fluid source.

The flexible gas-permeable hydrophobic membrane is suitably configured such that it can be at least partially deflected by pressure exerted through the first fluid channel. This may be accomplished by sealing the membrane to the substrate such that the membrane remains stationary (i.e., does not de-seal from the substrate), but deflects when sufficient pressure is exerted on the membrane by a pressure source (e.g., fluid) in the first fluid channel. This is illustrated by FIG. 1, which illustrates the upward deflection of the membrane when the fluid in the first fluid channel exerts sufficient pressure on the membrane that seals the outlet of the first fluid channel.

Debubblers may be a part of or connected to an analysis device. The debubbler may be inserted close to the device's inlet or in multiple positions within the device to remove bubbles introduced externally into the device, bubbles trapped between liquid streams, and bubbles formed during device's normal operation.

The debubblers can be used in conjuction with virtually any analysis device. A non-exclusive listing of such devices includes microarrays, bead arrays, packed beds, porous beds, reactors, mixers, PCR chambers, units for sample preparation, biosensors, chemical sensors, lab on chip devices, micro-total-analysis devices, and the like.

The fluid channels of the disclosed devices vary in size. The cross-sectional dimension (e.g., width, diameter) of the first fluid channel is suitably in the range of from about 1 micrometer to about 10 millimeters, or from about 5 micrometers to about 5 millimeters, or from micrometers to about 1 millimeter. The cross-sectional dimension of the membrane may be in the range of from about 0.01 mm to about 5 mm or even 5 cm, or from about 0.1 mm to about 1 mm, or even about 0.5 mm. The first and second fluid channels are suitably sized such that they can both be sealed by the membrane. Similarly, the membrane is suitably sized so as to seal the channels when the membrane is in a non-deflected configuration. The membrane may be circular in shape, and can also be oblong, polygonal, linear, or other shapes.

The cross sectional area of the first channel may be from about 0.1% to about 99%, or from about 5% to about 50%, or from 10% to about 20% of the surface area of the membrane that is exposed to the first channel. Likewise, the cross sectional area of the second channel may be from about 0.1% to about 99%, or from about 5% to about 50%, or from 10% to about 20% of the surface area of the membrane that is exposed to the second channel.

In some embodiments, the second fluid channel is in fluid communication with a gutter, and is not itself directly sealed by the membrane. In this embodiment, fluid exerted through the first fluid channel deflects the membrane, and fluid that exits the first fluid channel is “caught” by the gutter, which gutter is sealed by the membrane. The fluid is then carried by the gutter into the second fluid channel.

A debubbler may also suitably include a pressure source in fluid communication with the first fluid channel. The pressure source may be a pump, a syringe pump, a piston, a pinion drive, and the like. Virtually any device capable of exerting pressure on a fluid within a fluid channel is a suitable pressure source. The pressure source is suitably capable of exerting sufficient pressure on fluid contained within the first channel so as to deflect the flexible gas-permeable hydrophobic membrane by an amount sufficient to place the first and second fluid channels into fluid communication with one another. This is illustrated by FIG. 1, which depicts an embodiment where the pressure in the first fluid channel deflects the membrane upward such that fluid exiting the first channel is directed to the gutter and then to the second fluid channel.

Methods of degassing (e.g., debubbling) a fluid are also provided. These methods include exerting a fluid contained within a first fluid channel against a gas-permeable membrane sealing the first fluid channel so as to discharge a gas disposed in the first fluid channel through the membrane while the membrane remains essentially stationary. The user then pressurizes the fluid against the membrane so as to deflect the membrane, the deflection of the membrane placing the first fluid channel into fluid communication with a second fluid channel such that the fluid flows into the second fluid channel. The deflection of the membrane is suitably accomplished by application of sufficient pressure by the fluid contained within the first fluid channel.

The suitable fluid pressure will be determined by the user of ordinary skill without difficulty. The pressure may be in the range of from about 0.1 kPa to 50 kPa greater than ambient pressure, or from about 1 kPa to 10 kPa greater than ambient pressure. For the exemplary systems investigated herein, the pressure was in the range of about 4.7 kPa. Fluid may flow into the second channel at a rate of between about 0.01 microliters/minute to about 1000 microliters/minute, or about 0.1 microliters/minute to about 100 microliters/minute, or about 1 microliter/minute to about 10 microliters/minute, or even about 5 microliters/minute.

### Illustrative Embodiments

Provided here are robust passive microfluidic devices for rapid and efficient removal of air bubbles from liquid solutions, even when the liquids contain various surfactants. The disclosed devices were integrated the debubbler into an agarose bead array-based microfluidic cassette. The performance of the integrated cassette was examined by detecting haptenized PCR amplicons of B. Cereus bacteria in a sequential flow.

FIG. 1 depicts schematically the cross-section and the degassing principle of the debubbler. The debubbler consists of two essential components: a hydrophobic, porous, poly(tetrafluoro ethylene) (PTFE) venting membrane for rapid bubble removal and a conduit (nozzle) to direct the bubble-laden liquid towards the membrane. For illustrative embodiments, a device with poly(methyl methacrylate) (PMMA) was used. The 100 μm long, 330 μm inner diameter (ID), 1000 pm outer diameter (OD) nozzle was milled with a precision computer-controlled (CNC) milling machine (HAAS Automation Inc., Oxnard, Calif.).¹³ The PTFE membrane (5 pm pore size, Sterlitech Corporation, USA) was cut to a diameter of 2.5 mm with a Harris punch cutter (American MasterTech Scientific, Inc., Lodi, Calif.). The membrane was bonded against the nozzle with 100 μm thick, double-sided adhesive tape (3M Co., St. Paul, Minn., USA) that was patterned with a CO₂ laser (Universal Laser Systems Inc., USA). The suspended diameter of the membrane (D) in FIG. 1a is 1.2 mm.

Before the start of the degassing operation, the porous membrane pushes tightly against the nozzle's opening, and only air can flow freely through the membrane's pores (FIG. 1a). When liquid is delivered into the debubbler through its inlet, the air in the microchannel is discharged through the porous membrane to the ambient. When liquid pushes against the membrane (FIG. 1b), the membrane deforms and allows the passage of gas-free liquid beneath it and into the device. As long as the liquid pressure is not too high, the membrane is impermeable to liquid flow. The minimal pressure needed to deform the membrane is pₒₚₑₙ. When air bubbles migrate towards the membrane, the gas cannot maintain the pressure pₒₚₑₙ and the porous membrane recovers its closed, undeformed stage. The liquid pressure upstream forces the bubble to discharge through the pore of the hydrophobic membrane (FIG. 1c).

Debubbler Performance

The debubbler's efficiency was tested by introducing colored deionized (DI) water or phosphate buffered saline (PBS) blocking buffer (pH 7.4 3% (w/v) bovine serum albumin (BSA) and 0.1% Tween 20) into the microfluidic channel upstream of the debubbler and seeding the liquid with air bubbles (FIG. 1c). The bubble volume was estimated from the bubble's length and the known internal diameter of the tubing. The driving force was provided with a syringe pump (Model PHD 2000, Harvard Apparatus, Holliston, Mass.). Time-lapse images were recorded with a portable Sony digital camera (DCR-PC330, Japan). The time when the bubble entered the debubbler was set as t=0. To evaluate the pressure loss through the membrane-based debubbler, the liquid pressure at the debubbler's inlet was measured with a pressure sensor (model 26PCO1KOD6A, Sensortechnics Inc., USA) and the flow rate was calculated by measuring the volume discharged at the outlet within a preset time interval.

Bead Array-Based Cassette Integrated with Debubbler

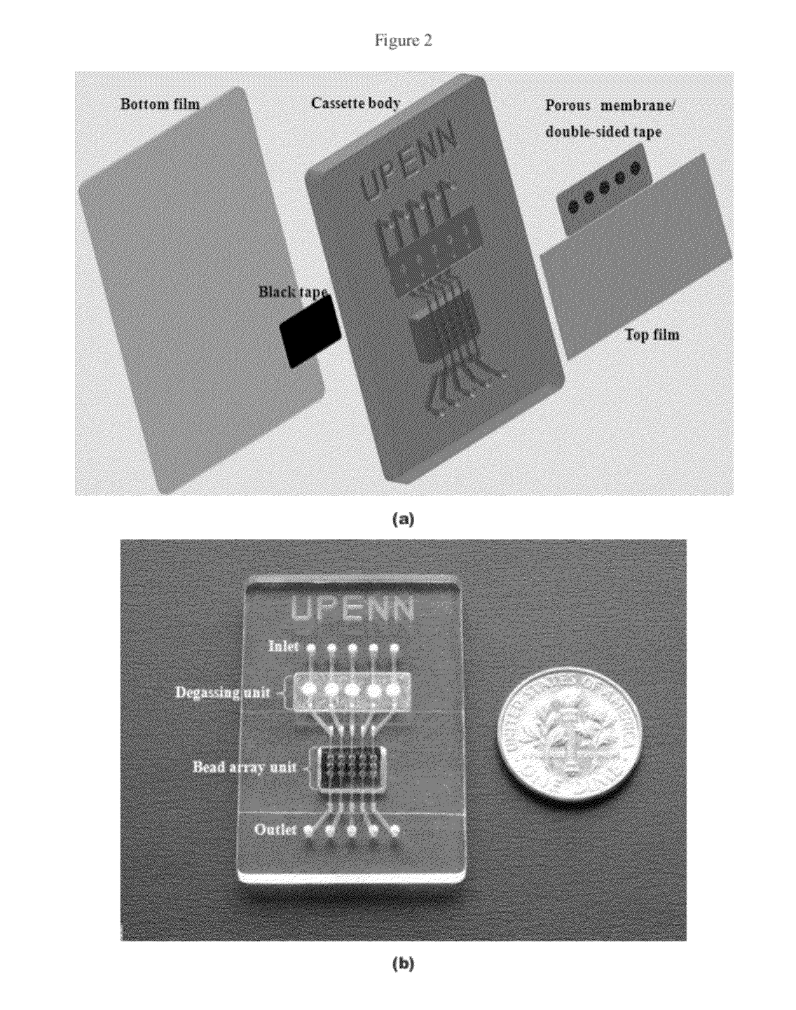

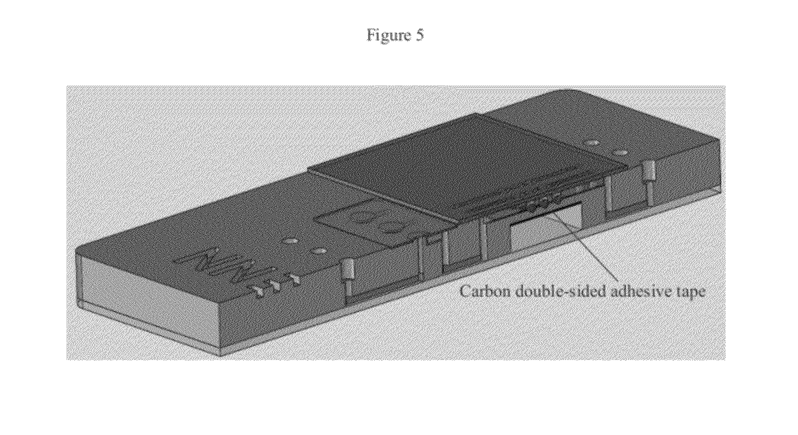

To evaluate the reliability, applicability, and compatibility of the debubbler for microfluidic applications, the debubbler was integrated with an agarose bead array-based microfluidic cassette and used the cassette to detect haptenized PCR amplicons of B. Cereus bacteria. FIG. 2a, FIG. 5, and FIG. 2b are, respectively, an exploded view, cross-sectional view, and a photograph of the integrated cassette. This 46 mm×36 mm×3.4 mm cassette has two major functional domains: a degassing unit and an agarose bead array unit for the capture of target analytes. All features, including nozzles, bead wells, and microchannels, were milled in the cassette's body using a CNC machine. The base of the cassette body was solvent-bonded to a 250 μm thick PMMA film at the room temperature. The degassing unit consists of five debubblers, each connected to an independent linear microchannel leading downstream to the bead array unit (FIG. 2b).

Each bead array unit has three wells (600 pm diameter×650 pm deep) along each of five adjacent channels (330 pm width×300 pm depth). Each microfabricated well holds a single, 500 pm diameter, sbeptavidin-coated agarose bead. The plastic substrate beneath the beads was thinned down and coated with a carbon black tape to minimize background fluorescent emission (FIG. 5). When sample or buffer was introduced into the cassette from the inlet, it was first degassed by the upstream debubbler, and then delivered to the beads where targets could be captured and imaged.

Detection of PCR Products of B. cereus Bacteria

Bacillus cereus is a Gram-positive bacteria that produces toxins, which may cause food poisoning. As a model analyte, the double-labeled amplicons of B. cereus genomic DNA templates were detected in the bead array-based microfluidic cassette with the integrated debubblers. Streptavidin docicing sites were coupled to the aldehyde moiety of a glyoxylated agarose bead (BioScience Bead Division of CSS, West Warwick, R1) via reductive amination. The DNA assay on the streptavidin-coated agarose beads consisted of five sequential steps: (i) a sample containing haptenized DNA amplicons suspended in PBS buffer was delivered into the cassette at a flow rate of 10 μ/min for 2 min and incubated with the beads for 3 min at room temperature. The method of haptenizing the amplicons has been described previously. (ii) The beads were washed with 0.3 mL of PBS buffer at a flow rate of 30 μ/min to remove any unbound DNA. (iii) The beads were blocked with PBS blocking buffer containing 3% BSA and 0.1% Tween-20 for 10 min at a flow rate of 30 μ/min. (iv) Anti-digoxigenin-fluorescein complex suspended in PBS buffer (150 dilution in PBS) (Roche Diagnostics, Indianapolis, Ind.) was injected into the cassette at a flow rate of 10 μ/min for 3 min and incubated for 10 min with the beads. (v) The beads were washed with 0.3 mL of PBS buffer at a flow rate of 30 μ/min to remove any unbound anti-digoxigenin-fluorescein complex. The flow rates and incubation times were not optimized. This particular assay was selected for study because the numerous switching among various solutions provide ample opportunity for bubble entrapment.

Fluorescent images of the bead array were acquired with an Olympus BX5 1 microscope, equipped with various objectives, a filter cube (480 nm excitation, 505 long-pass beam splitter dichroic mirror, and 535±25 nm emission), a charge-coupled device (CCD) camera (PCO imaging, Germany), and a mercury discharge lamp light source. Areas of interest in the array were selected to monitor emission intensities. The data was analyzed with ImageJ analysis software (National institutes of Health, Bethesda, Md.).

Results

PTFE was selected as the degassing membrane due to its flexibility and high hydrophobicity. In its closed state, the membrane pushes tightly against the debubbler's nozzle. As long as the liquid pressure does not exceed a certain value, the membrane acts as a semi-permeable valve.

There are two pressures that control the operation of the debubbler. pₒₚₑₙ is the minimal pressure difference between the liquid pressure (p₁) and the ambient pressure (p₀) that is needed to deflect the membrane and allow the liquid to flow from the inlet conduit to the outlet conduit (FIG. 1b). As long as p=(p₁−p₀)<pₒₚₑₙ, liquid cannot reach the outlet conduit of the debubbler. The magnitude of pₒₚₑₙ is dictated by the suspended diameter (D), thickness, pretension, and elastic properties of the membrane, and the nozzle diameter. For the exemplary debubbler, pₒₚₑₙ=4.71±0.95 kPa (n=5).

Once the liquid contacts the porous membrane in the debubbler, a meniscus forms at the entrance corner of a hydrophobic venting pore (see inset of FIG. 1b). This meniscus can change shape to accommodate the applied pressure difference. Although liquid will not enter spontaneously into the hydrophobic pore, external pressure may force it to enter. At equilibrium, according to the Laplace-Young equation:

p₁−p₀=4γ cos(180−θ)/d, (1)

where d is the diameter of the pore, γ is the surface tension at the liquid-air interface (γ=72.75×10⁻³ N·m⁻¹ for pure water), and θ is the angle between the meniscus and the pore's surface. As p₁ increases, θ increases until it exceeds the critical value θₘₐₓ. Once θₘₐₓ is exceeded, the liquid will leak through the pore. Therefore, the maximum pressure difference (Δp)ₘₐₓ that the hydrophobic capillary can withstand (leakage onset pressure pₗₑₐₖ) is:

pₗₑₐₖ=(Δp)ₘₐₓ=4γ cos(180−θₘₐₓ)/d, (2)

where the maximum value θₘₐₓ is equal to the advancing contact angle θ_{adv}, which is 115° between pure water and PTFE. When the PTFE membrane pore's diameter is 5 μm and the working fluid is DI water, the theoretical leakage onset pressure pₗₑₐₖ is 24.6 kPa. In these experiments, leakage pressure of 25.2±4.3 kPa (n=5) was measured, which is in good agreement with the theoretical estimate.

For some operations of the debubbler, gas is vented without any liquid leakage; that is, (p₁−p₀)<pₗₑₐₖ. The leakage onset pressure of 25 kPa was adequate. Larger leakage onset pressures can be attained witrough the debubbler, the liquid is, of course, subject to evaporation at the liquid/air interface, but the rate of evaporation is deemed negligible given the small cross-sectional area of the pores.

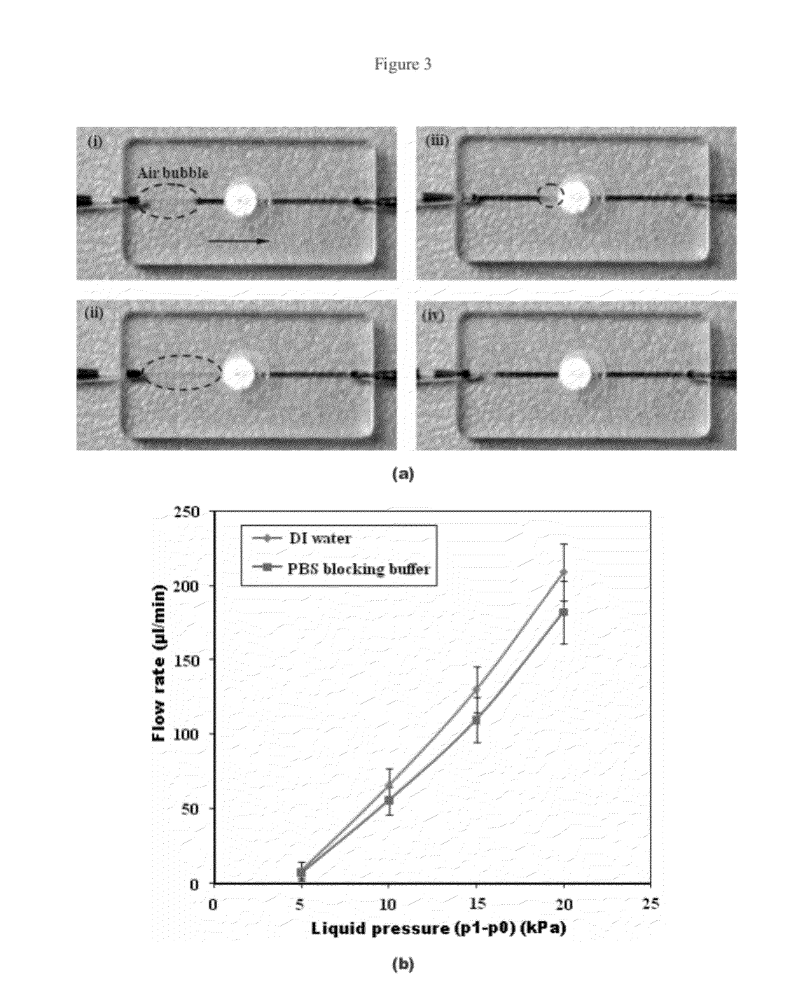

In many microfluidic experiments, surfactants, proteins, and salts are often contained in the liquid. The presence of surfactants and proteins can not only reduce the contact angle of the liquid, but also stabilize the bubble film, rendering bubble removal more difficult. The device was evaluated by watching for the presence of air bubbles in the outlet of the debubbler. The image sequence in FIG. 3(a) demonstrates the bubble dynamics in DI water during the removal process when the flow rate is 200 μl/min. An air bubble enters the debubbler (FIG. 3a i) and migrates towards the degsassing membrane (FIG. 3a ii).

Once the air bubble reaches the membrane (FIG. 3a iii), it permeates through the membrane. Downstream of the membrane, the fluid is completely bubble-free (FIG. 3a iv). Leakage of DI water and PBS blocking buffer through the membrane was found to occur, respectively, at flow rates of 310±21 μl/min (n=5) and 275±17 μl/min (n=5). Complete gas extraction was achieved for a maximum degassing rate of about 60 μl/s/mm² in DI water with a 5 μm pore-sized PTFE membrane.

The pressure loss in the debubbler was also measured. FIG. 3(b) depicts the flow rates of DI water and PBS blocking buffer through the debubbler as functions of liquid pressure (p₁−p₀) at the debubbler's inlet. As long as the inlet pressure is below the threshold pressure, the membrane acts as a normally closed valve and there is no flow through the debubbler. Once the threshold pressure is exceeded, the flow rate increases slightly faster than linearly.

DNA Detection on Integrated Cassette

Bead array-based microfluidic chips have been widely used in many bioanalytical applications due to their high throughput, low consumption of samples and reagents, and high sensitivity. These devices contain, however, wells and features that can easily trap air bubbles. Once trapped, the air bubbles accumulate, adversely affect device performance, and are very difficult to remove. Too high a flow rate not only wastes expensive biological reagents, but also may deform the soft agarose beads as well as adversely affect biological interactions. Here, the debubbler is incorporated into a bead array-based cassette for rapid bubble removal under normal microfluidic operation.

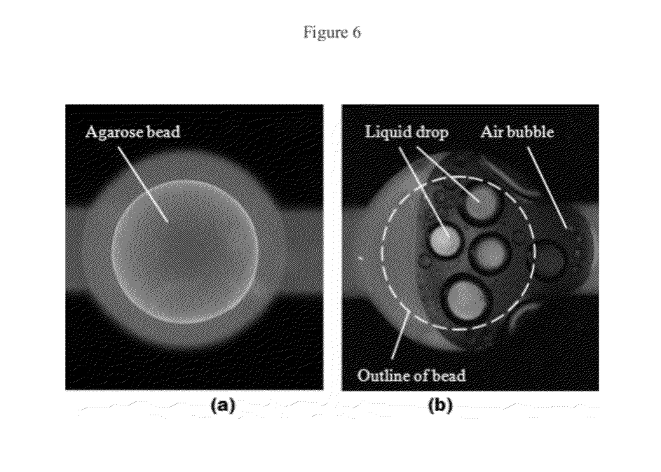

FIG. 6 shows a fluorescent image of an agarose bead for DNA detection in a bead array-based cassette integrated with debubblers. Air bubbles introduced upstream were successfully prevented from migrating into the bead wells, verifying the efficiency of the debubbler. In contrast, in the absence of a debubbler, a large bubble was trapped in the bead well, which interfered with the fluorescent signal acquisition as well as reagent transport to the bead surface (FIG. 6).

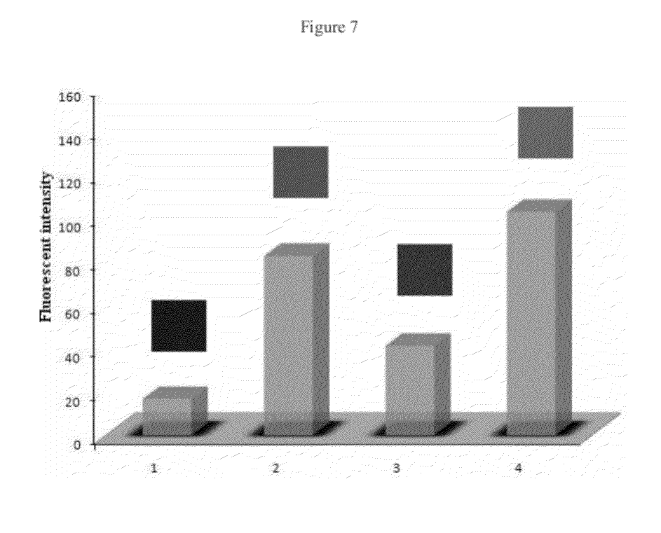

In one embodiment, the thickness of the PMMA substrate was reduced to 0.8 mm without sacrificing the structural integrity of the cassette by milling a 8 mm long×5 mm wide×2.2 mm deep chamber beneath the agarose bead array (FIG. 5) (PMMA may in some cases exhibit background fluorescence, but is still considered a suitable material for use in the debubblers). To further reduce interference from external sources, a black, low background fluorescence, carbon, double-sided adhesive tape was attached to the milled chamber (FIG. 7).

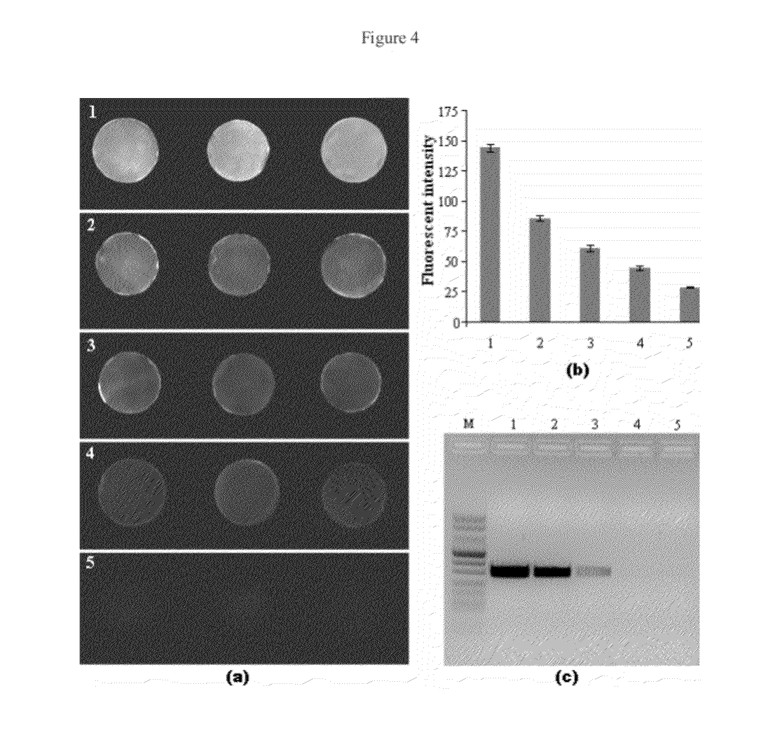

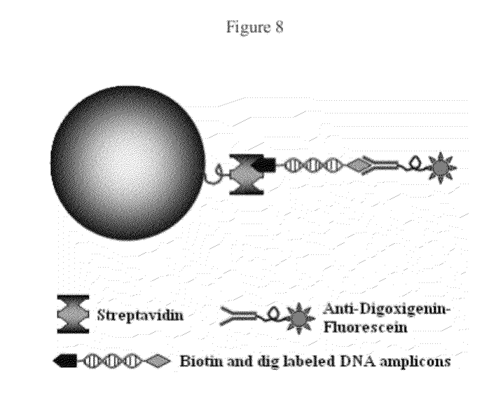

To demonstrate the effectiveness of the bead array with the integrated bubbler, the array was used to detect PCR-amplified B. Cereus DNA sequences of 305-bp length. To this end, the primers were haptenized with biotin and digoxigenin (dig). As a result, the PCR amplification products were functionalized with biotin and dig. The B. Cereus DNA amplicons bonded to the streptavidin-coated agarose bead in the cassette through their biotin functionalization and the label bonded to the amplicon via the dig functionalization. FIG. 8 depicts the operating principle of the streptavidin-coated agarose bead assay. The fluorescent signal depended on the amount of the bound fluorescein complexes. FIG. 4a is a sequence of fluorescent images of the beads with different PCR amplicon concentrations obtained from samples containing B. Cereus DNA templates ranging in mass from 0 to 10 ng. In the presence of the upstream debubbler, no air bubbles were observed in the bead wells. FIG. 4b depicts the measured fluorescent intensity of the beads as a function of the B. Cereus DNA template concentration (prior to amplification). Bars 1, 2, 3, 4, 5 correspond, respectively, to DNA template of masses of 10, 1, 0.1, 0.01, and 0 (negative control) ng (n=6). FIG. 4c is a gel electropherogram of the various PCR amplicons. Lane M is the DNA marker VIII (Roche Diagnostics). Lanes 1-5 should be cross-referenced with columns 1-5 in FIG. 4b. FIG. 4 clearly demonstrates that the accumulation of bubbles was successfully prevented. The cassette could detect amplicons of 10 pg DNA template of B. Cereus, which exceeds the detection ability of conventional gel electrophoresis by approximately a factor of 10.

Additional Discussion

The disclosed debubblers are readily integrated upstream of bubble-sensitive, microfluidic modules. This allows the modules to operate properly even when the flow stream entering the device is laden with gas bubbles. The debubbler allows for rapid and complete degassing. The debubbler removes efficiently bubbles with a broad range of sizes. The device requires a pressure source, and can operate with pure water as well as with buffers containing surfactants.

In some embodiments (not shown), multiple debubblers are incorporated into a microfluidic system. This may be done to facilitate gas purging between various unit operations. For example, a debubbler may be incorporated at the inlet of the analysis system so as to degas fluid that enters the system. A second debubbler may be present between a reactor module and a postprocessing module (or other fluid elements) so as to remove gas that may evolve during a reaction that takes place in the reactor module. A further debubbler may be positioned at the outlet of the system so as to degas fluid that exits the system. The debubblers may be integrated into the system at the time of manufacture. Debubblers may also fabricated as modules or chips that can be inserted into a microfluidic system.

To demonstrate the debubblers' usefulness, the debubbler was incorporated into a bead array-based microfluidic cassette, which was used to detect haptenized PCR amplicons of B. Cereus bacteria. The bead array outperformed conventional gel electrophoresis. The proposed debubbler can also work as an independent or integrated module in a variety of other microfluidic flow devices.

What is claimed:

1. A debubbler, comprising:
a substrate having at least one of a first fluid channel and a second fluid channel formed therein; and
a flexible gas-permeable hydrophobic membrane sealing an outlet of the first fluid channel,
the first and second fluid channels being in fluid communication with one another when the flexible gas-permeable hydrophobic membrane is in a deflected state.

2. The debubbler of claim 1, wherein the flexible, gas-permeable hydrophobic membrane is porous.

3. The debubbler of claim 2, wherein the flexible gas-permeable hydrophobic membrane comprises polytetafluoroethylene, polyvinylidene fluoride, polypropylene, polyethylene, acrylic polymer, or any combination thereof.

4. The debubbler of claim 1, wherein the flexible, gas-permeable, hydrophobic membrane is essentially impermeable to liquids when the liquid pressure is below a certain threshold.

5. The debubbler of claim 1, wherein the flexible gas-permeable hydrophobic membrane is glued to the substrate, taped to the substrate, welded to the substrate, bonded to the substrate, or any combination thereof.

6. The debubbler of claim 1, wherein the flexible gas-permeable membrane is molded into the substrate.

7. The debubbler of claim 1, wherein the flexible gas-permeable hydrophobic membrane contacts a raised portion of the substrate.

8. The debubbler of claim 7, wherein at least one of the first fluid channel or the second fluid channel is formed in the raised portion of the substrate.

9. The debubbler of claim 1, wherein the membrane and substrate define a gutter capable of fluid communication with the second fluid channel.

10. The debubbler of claim 1, further comprising a fluid reservoir in fluid communication with the first fluid channel.

11. The debubbler of claim 1, wherein the flexible gas-permeable hydrophobic membrane is configured such that it can be at least partially deflected by pressure exerted through the first fluid channel.

12. The debubbler of claim 1, further comprising an inlet placing the debubbler into fluid communication with the environment exterior to the debubbler.

13. The debubbler of claim 1, wherein the membrane is in fluid communications with two or more inlets.

14. The debubbler of claim 1, wherein the membrane is in fluid communication with two or more outlet conduits.

15. The debubbler of claim 1, further comprising an analysis device in fluid communication with at least one of the first fluid channel or the second fluid channel of the debubbler.

16. The debubbler of claim 15, wherein the analysis device comprises a microarray, a bead array, a functionalized surface, a packed bed, a porous bed, reaction chamber, mixing chamber, amplification chamber, detection chamber, lab on chip, micro total analysis system, diagnostic device, or any combination thereof.

17. The debubbler of claim 1, wherein the cross-sectional dimension of the first fluid channel is in the range of from about 1 micrometer to about 3 millimeters.

18. The debubbler of claim 1, wherein the membrane has a cross-sectional dimension in the range of from about 0.01 mm to about 5 mm.

19. The debubbler of claim 1, further comprising a pressure source in fluid communication with the first fluid channel, the pressure source being capable of exerting sufficient pressure on fluid contained within the first channel so as to deflect the flexible gas-permeable hydrophobic membrane by an amount sufficient to place the first and second fluid channels into fluid communication with one another.

20. A method of degassing a fluid, comprising:
exerting a fluid contained within a first fluid channel against a gas-permeable membrane sealing the first fluid channel so as to discharge a gas disposed in the first fluid channel through the membrane while the membrane remains essentially stationary;
exerting the fluid against the membrane so as to deflect the membrane,
the deflection of the membrane placing the first fluid channel into fluid communication with a second fluid channel such that the fluid flows into the second fluid channel.
